(19) United States
(12) Patent Application Publication
(10) Pub. No.: US 2023/0372632 A1
(43) Pub. Date: Nov. 23, 2023

(54) DRIVING MECHANISM FOR DRIVING A PLUNGER OF AN AUTO-INJECTOR TO SLIDE RELATIVE TO A RESERVOIR OF THE AUTO-INJECTOR AND AUTO-INJECTOR THEREWITH

(71) Applicant: ALTEK BIOTECHNOLOGY CORPORATION, Hsinchu City (TW)

(72) Inventor: Yu-Cheng Huang, Hsinchu City (TW)

(73) Assignee: ALTEK BIOTECHNOLOGY CORPORATION, Hsinchu City (TW)

(21) Appl. No.: 18/125,129

(22) Filed: Mar. 23, 2023

Related U.S. Application Data
(60) Provisional application No. 63/344,631, filed on May 22, 2022.

(51) Int. Cl. A61M 5/315; A61M 5/20

(52) U.S. Cl. CPC A61M 5/31583; A61M 5/20; A61M 5/31513; A61M 2205/3327

(57) ABSTRACT

A driving mechanism for driving a plunger of an auto-injector to slide relative to a reservoir of the auto-injector is provided and includes a first transmission component, a driving component, a second transmission component, a driving resilient component, a stopping resilient component, a third transmission component, a sliding component and a supporting component. The second transmission component resiliently deforms the stopping resilient component when the first transmission component resiliently deforms the driving resilient component to push the second transmission component to rotate along a first rotating direction. The second transmission component is stopped from rotating along a second rotating direction by the stopping resilient component when the driving resilient component is released to resiliently recover. The third transmission component drives the sliding component to slide when the second transmission component rotates. The supporting component guides the sliding component to slide. Besides, a related auto-injector is provided.

### CROSS REFERENCE TO RELATED APPLICATIONS

This application claims the benefit of U.S. Provisional Application No. 63/344,631, filed on May 22, 2022. The content of the application is incorporated herein by reference.

### BACKGROUND OF THE INVENTION

### 1. Field of the Invention

The present invention relates to a driving mechanism and an auto-injector therewith, and more specifically, to a driving mechanism for driving a plunger of an auto-injector to slide relative to a reservoir of the auto-injector and an auto-injector therewith.

### 2. Description of the Prior Art

An auto-injector, e.g., an on-body injector, is a medical device designed to deliver a dose of a drug. However, the conventional auto-injectors available in the markets are unable to meet requirements of small volume, high driving power, long driving distance, long injecting period and accurate drug dose delivery rate. Therefore, an improvement of the auto-injector is urgently needed.

### SUMMARY OF THE INVENTION

Therefore, it is an objective of the present invention to provide a driving mechanism for driving a plunger of an auto-injector to slide relative to a reservoir of the auto-injector and an auto-injector therewith for solving the aforementioned problems.

In order to achieve the aforementioned objective, the present invention discloses a driving mechanism for driving a plunger of an auto-injector to slide relative to a reservoir of the auto-injector. The driving mechanism includes a first transmission component, a driving component, a second transmission component, a driving resilient component, a stopping resilient component, a third transmission component, a sliding component and a supporting component. The driving component is coupled to the first transmission component and for driving the first transmission component to rotate. The second transmission component is rotatably disposed apart from the first transmission component. The driving resilient component is arranged between the first transmission component and the second transmission component. The driving resilient component is forced by the first transmission component to resiliently deform to push the second transmission component to rotate along a first rotating direction or released to resiliently cover. The stopping resilient component is disposed adjacent to an outer periphery of the second transmission component. The stopping resilient component is forced by the second transmission component to resiliently deform when the driving resilient component is forced by the first transmission component to resiliently deform to push the second transmission component to rotate along the first rotating direction. The stopping resilient component engages with the second transmission component for stopping the second transmission component from rotating along a second rotating direction opposite to the first rotating direction when the driving resilient component is released to resiliently recover. The third transmission component is fixedly connected to the second transmission component. The third transmission component is driven by the second transmission component to rotate together with the second transmission component when the second transmission component rotates. The sliding component is at least partially slidably disposed inside the third transmission component and movably engaged with the third transmission component. The sliding component is connected to the plunger. The sliding component is driven by the third transmission component to slide relative to the second transmission component along a first sliding direction when the third transmission component is driven by the second transmission component to rotate along the first sliding direction together with the second transmission component. The supporting component includes a guiding portion. The sliding component passes through the guiding portion, and the guiding portion is configured to guide the sliding component to slide along the first sliding direction without rotation.

According to an embodiment of the present invention, the driving mechanism further includes a sensor configured to sense a rotating movement of the second transmission component.

According to an embodiment of the present invention, the sensor includes an abutting component. The abutting component is abutted by the stopping resilient component when the stopping resilient component is resiliently deformed by the second transmission component.

According to an embodiment of the present invention, the driving resilient component and the stopping resilient component are integrally connected to each other to form an integral resilient structure.

According to an embodiment of the present invention, the supporting component further includes a mounting portion for mounting the integral resilient structure.

According to an embodiment of the present invention, a channel is formed on the mounting portion. The integral resilient structure passes through the channel, and the driving resilient component and the stopping resilient component are partially exposed out of the mounting portion.

According to an embodiment of the present invention, the driving resilient component is resiliently deformed by the first transmission component along a first deforming direction identical to the first rotating direction, and the stopping resilient component is resiliently deformed by the second transmission component along a second deforming direction opposite to the first deforming direction.

According to an embodiment of the present invention, the first transmission component, the second transmission component and the third transmission component are accommodated inside the supporting component.

According to an embodiment of the present invention, a first rotating axis of the first transmission component is parallel to a second rotating axis of the second transmission component, and the first sliding direction is parallel to an extending direction of the second rotating axis of the second transmission component.

According to an embodiment of the present invention, the first transmission component is a cam component. The second transmission component is a ratchet component. The third transmission component is a screw sleeve. The sliding component is a screw rod, and the driving component is an electric motor.

According to an embodiment of the present invention, the driving mechanism further includes a reducer coupled between the driving component and the first transmission component.

According to an embodiment of the present invention, the reducer is a gearbox.

According to an embodiment of the present invention, the guiding portion includes a sliding through hole structure. The sliding component slidably passes through the sliding through hole structure. A cross section of the sliding component matches with a cross section of the sliding through hole structure. The sliding component includes at least one first arc part and at least one first flat part connected to the at least one first arc part. The sliding through hole structure includes at least one second arc part and at least one second flat part connected to the at least one second arc part, and the at least one second arc part and the at least one second flat part are arranged respectively corresponding to the at least one first arc part and the at least one first flat part.

According to an embodiment of the present invention, an internal thread structure is formed on an inner periphery of the third transmission component, and an external thread structure is formed on the at least one first arc part of the sliding component.

In order to achieve the aforementioned objective, the present invention further discloses an auto-injector. The auto-injector includes a reservoir, a plunger and a driving mechanism. The plunger is slidably disposed inside the reservoir. The driving mechanism is for driving the plunger to slide relative to the reservoir. The driving mechanism includes a first transmission component, a driving component, a second transmission component, a driving resilient component, a stopping resilient component, a third transmission component, a sliding component and a supporting component. The driving component is coupled to the first transmission component and for driving the first transmission component to rotate. The second transmission component is rotatably disposed apart from the first transmission component. The driving resilient component is arranged between the first transmission component and the second transmission component. The driving resilient component is forced by the first transmission component to resiliently deform to push the second transmission component to rotate along a first rotating direction or released to resiliently cover. The stopping resilient component is disposed adjacent to an outer periphery of the second transmission component. The stopping resilient component is forced by the second transmission component to resiliently deform when the driving resilient component is forced by the first transmission component to resiliently deform to push the second transmission component to rotate along the first rotating direction. The stopping resilient component engages with the second transmission component for stopping the second transmission component from rotating along a second rotating direction opposite to the first rotating direction when the driving resilient component is released to resiliently recover. The third transmission component is fixedly connected to the second transmission component. The third transmission component is driven by the second transmission component to rotate together with the second transmission component when the second transmission component rotates. The sliding component is at least partially slidably disposed inside the third transmission component and movably engaged with the third transmission component. The sliding component is connected to the plunger. The sliding component is driven by the third transmission component to slide relative to the second transmission component along a first sliding direction when the third transmission component is driven by the second transmission component to rotate along the first sliding direction together with the second transmission component. The supporting component includes a guiding portion. The sliding component passes through the guiding portion, and the guiding portion is configured to guide the sliding component to slide along the first sliding direction without rotation.

According to an embodiment of the present invention, the driving mechanism further includes a sensor configured to sense a rotating movement of the second transmission component.

According to an embodiment of the present invention, the sensor includes an abutting component. The abutting component is abutted by the stopping resilient component when the stopping resilient component is resiliently deformed by the second transmission component.

According to an embodiment of the present invention, the driving resilient component and the stopping resilient component are integrally connected to each other to form an integral resilient structure, and the supporting component further comprises a mounting portion for mounting the integral resilient structure.

According to an embodiment of the present invention, a channel is formed on the mounting portion. The integral resilient structure passes through the channel, and the driving resilient component and the stopping resilient component are partially exposed out of the mounting portion.

According to an embodiment of the present invention, the driving resilient component is resiliently deformed by the first transmission component along a first deforming direction identical to the first rotating direction, and the stopping resilient component is resiliently deformed by the second transmission component along a second deforming direction opposite to the first deforming direction.

In summary, in the present invention, the driving mechanism not only has compact structure and high power and high efficiency transmission but also achieves a long sliding distance and a slow sliding speed of the sliding component and prevents any rotation of the sliding component during a sliding movement of the sliding component. Therefore, the present invention can meet requirements of small volume, high driving power, long driving distance, long injecting period and accurate drug dose delivery rate.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are partial diagrams of an auto-injector at different views according to a first embodiment of the present invention.

FIG. 3 and FIG. 4 are diagrams of a driving mechanism at different views according to the first embodiment of the present invention.

FIG. 5 and FIG. 6 are partial diagrams of the driving mechanism at different views according to the first embodiment of the present invention.

FIG. 7 is another partial diagram of the driving mechanism according to the first embodiment of the present invention.

FIG. 8 and FIG. 9 are exploded diagrams of the driving mechanism at different views according to the first embodiment of the present invention.

FIG. 10 is a partial enlarged diagram of the driving mechanism according to the first embodiment of the present invention.

FIG. 11 is a functional block diagram of the driving mechanism according to the first embodiment of the present invention.

FIG. 12 and FIG. 13 are diagrams of the driving mechanism in different states according to the first embodiment of the present invention.

FIG. 14 is a diagram of a first transmission component according to a second embodiment of the present invention.

FIG. 15 is a diagram of a first transmission component according to a third embodiment of the present invention.

### DETAILED DESCRIPTION

In the following detailed description of the preferred embodiments, reference is made to the accompanying drawings which form a part hereof, and in which is shown by way of illustration specific embodiments in which the invention may be practiced. In this regard, directional terminology, such as “top”, “bottom”, “left”, “right”, “front”, “back”, etc., is used with reference to the orientation of the Figure(s) being described. The components of the present invention can be positioned in a number of different orientations. As such, the directional terminology is used for purposes of illustration and is in no way limiting. Accordingly, the drawings and descriptions will be regarded as illustrative in nature and not as restrictive. Also, if not specified, the term “connect” or “couple” is intended to mean either an indirect or direct electrical/mechanical connection. Thus, if a first device is connected or coupled to a second device, that connection may be through a direct electrical/mechanical connection, or through an indirect electrical/mechanical connection via other devices and connections.

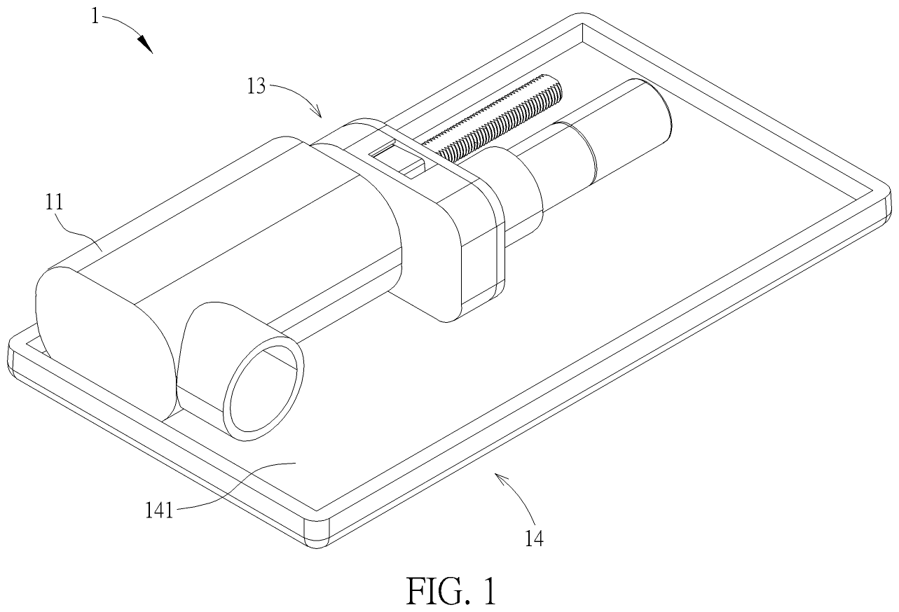

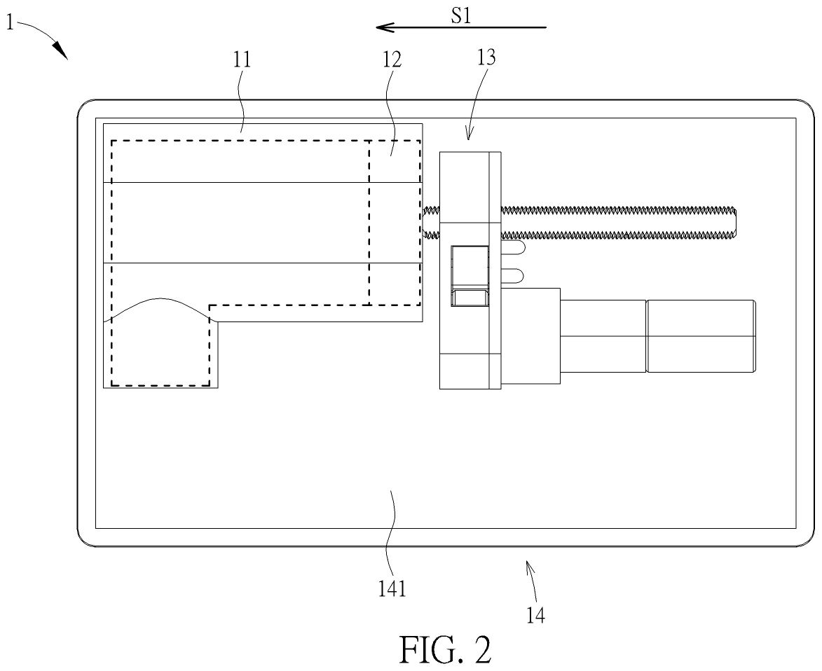

Please refer to FIG. 1 and FIG. 2. FIG. 1 and FIG. 2 are partial diagrams of an auto-injector 1 at different views according to a first embodiment of the present invention. As shown in FIG. 1 and FIG. 2, the auto-injector 1 includes a reservoir 11, a plunger 12 and a driving mechanism 13. The reservoir 11 is for drug storage. The plunger 12 is slidably disposed inside the reservoir 11. The driving mechanism 13 is for driving the plunger 12 to slide relative to the reservoir 11, so as to pump drug out of the reservoir 11 to complete a drug injection.

In this embodiment, the auto-injector 1 further includes a case 14. The case 14 includes a first mounting part 141, e.g., a lower shell, and a second mounting part, e.g., an upper shell, which is not shown in the figures, detachably installed on the first mounting part 141. The reservoir 11 and the driving mechanism 13 are mounted on the first mounting part 141. The case 14 is configured to conceal the reservoir 11 and the driving mechanism 13 for preventing damage of the reservoir 11 and the driving mechanism 13.

However, the present invention is not limited to this embodiment. For example, in another embodiment, the case can be a one-piece structure with an opening to at least partially reveal the reservoir and the driving mechanism.

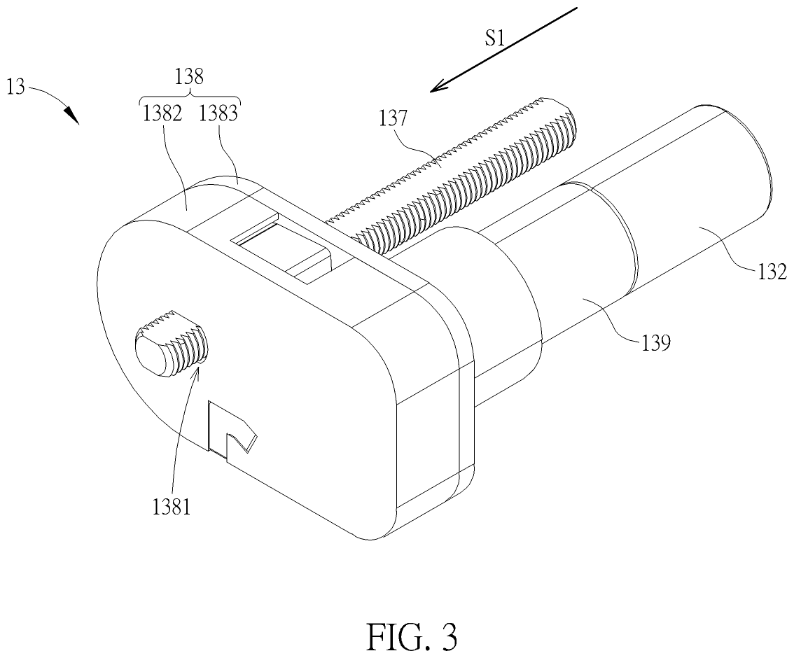

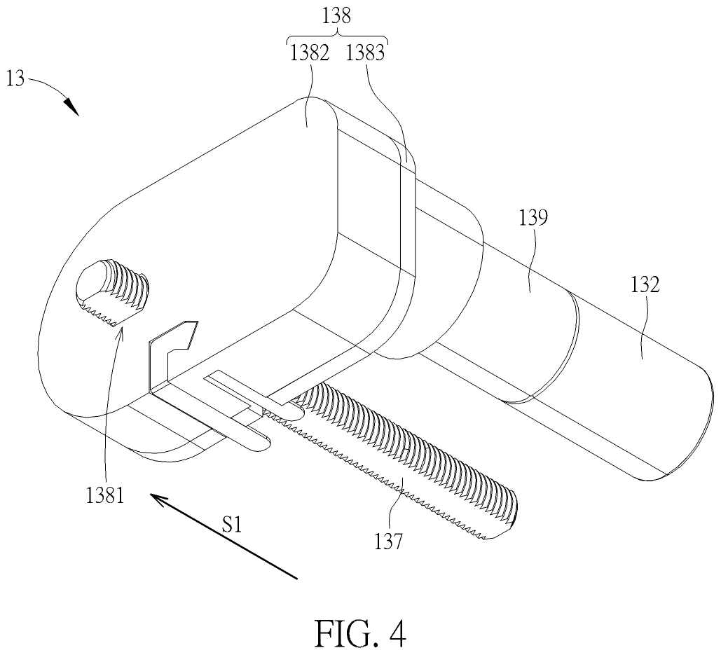

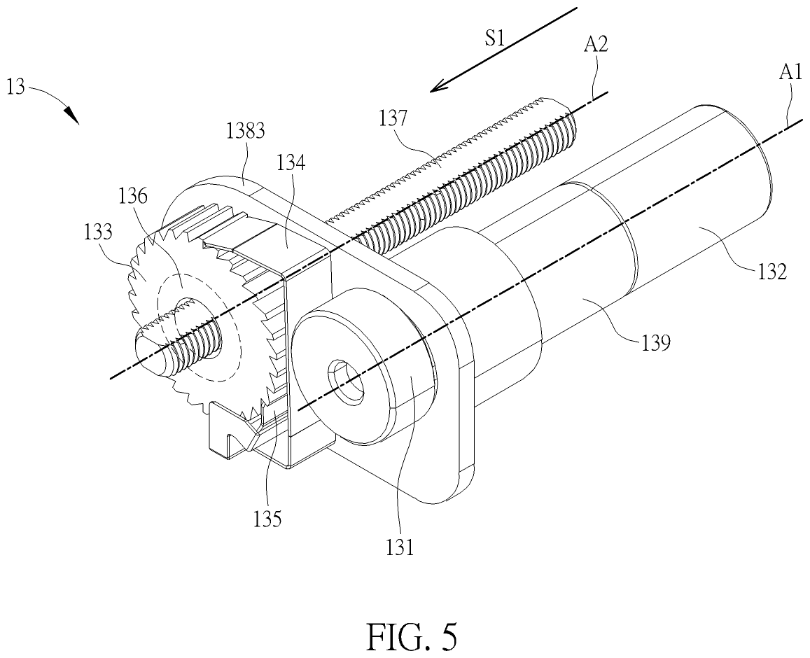

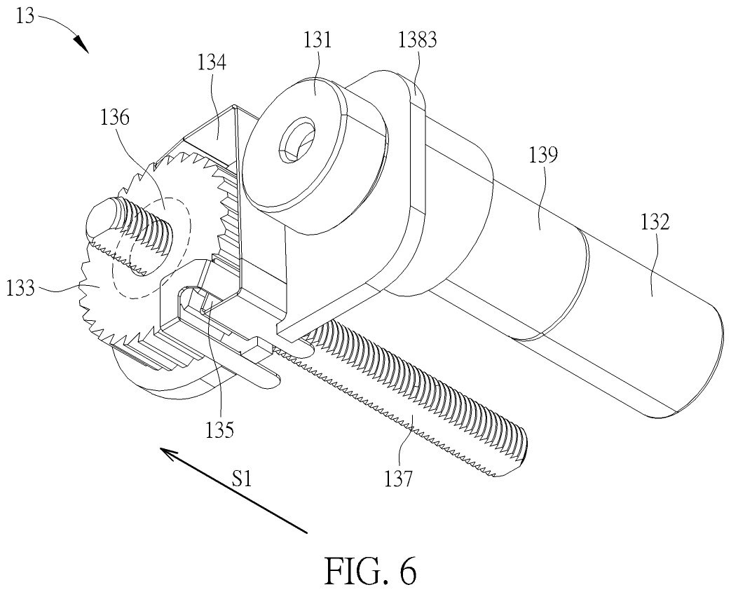

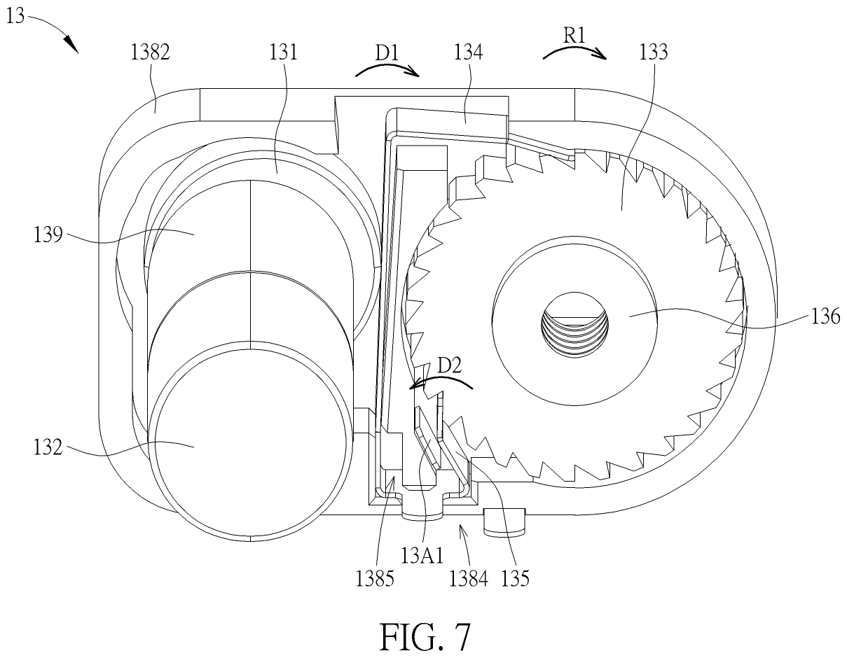

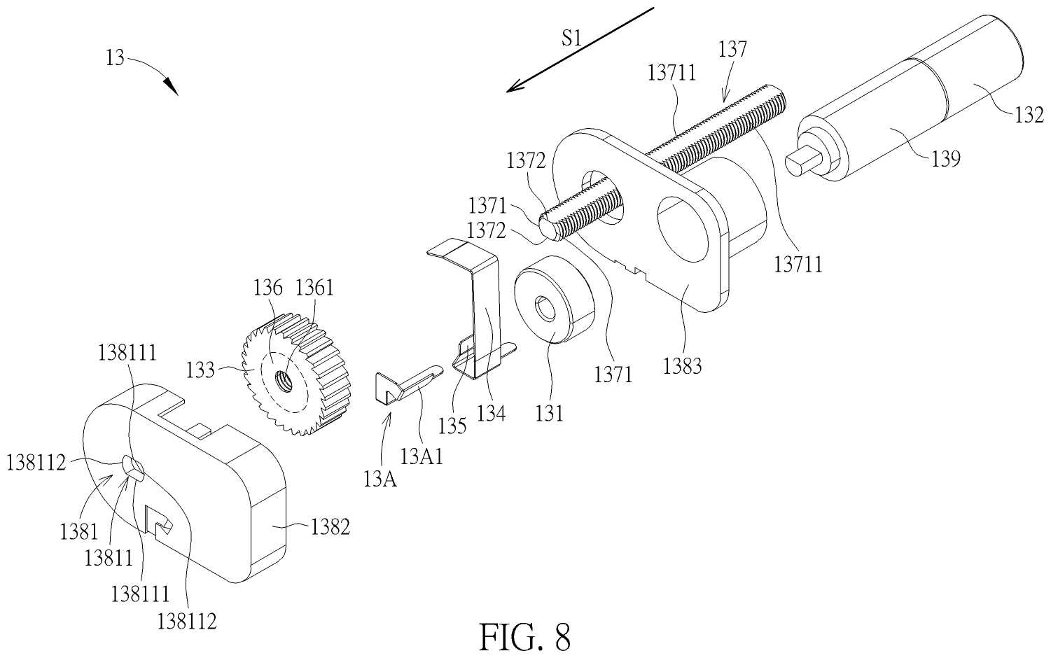

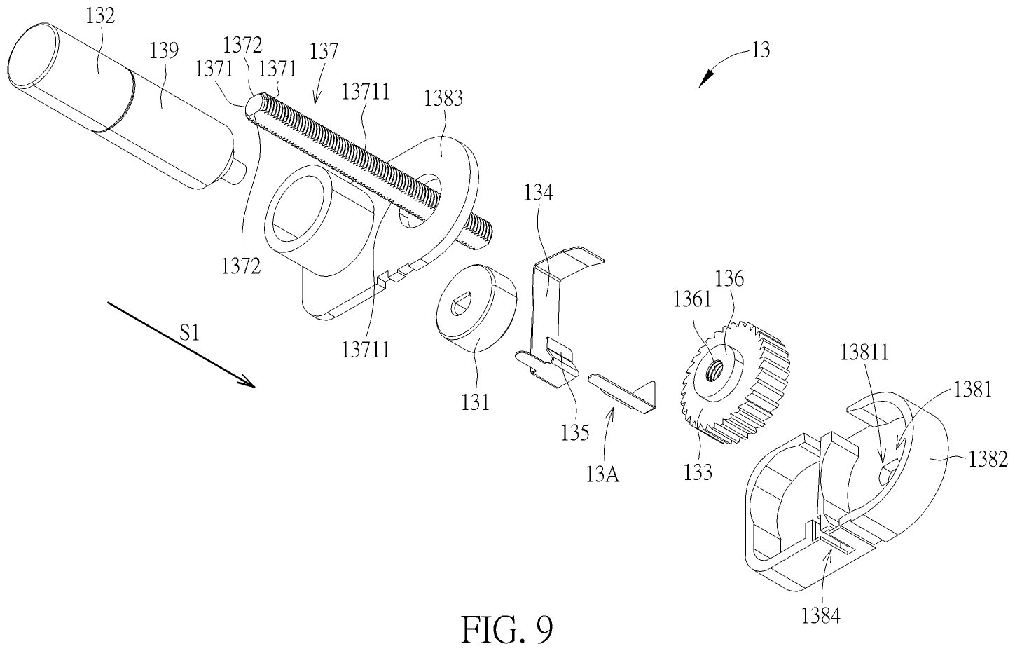

Please refer to FIG. 3 to FIG. 9. FIG. 3 and FIG. 4 are diagrams of the driving mechanism 13 at different views according to the first embodiment of the present invention. FIG. 5 and FIG. 6 are partial diagrams of the driving mechanism 13 at different views according to the first embodiment of the present invention. FIG. 7 is another partial diagram of the driving mechanism 13 according to the first embodiment of the present invention. FIG. 8 and FIG. 9 are exploded diagrams of the driving mechanism 13 at different views according to the first embodiment of the present invention. As shown in FIG. 3 to FIG. 9, the driving mechanism 13 includes a first transmission component 131, a driving component 132, a second transmission component 133, a driving resilient component 134, a stopping resilient component 135, a third transmission component 136, a sliding component 137 and a supporting component 138. The driving component 132 is coupled to the first transmission component 131 and for driving the first transmission component 131 to rotate. The second transmission component 133 is rotatably disposed apart from the first transmission component 131. The driving resilient component 134 is arranged between the first transmission component 131 and the second transmission component 133. The driving resilient component 134 is configured to be forced by the first transmission component 131 to resiliently deform to push the second transmission component 133 to rotate along a first rotating direction R1, or released to resiliently cover. The stopping resilient component 135 is disposed adjacent to an outer periphery of the second transmission component 133. The stopping resilient component 135 is configured to be forced by the second transmission component 133 to resiliently deform when the driving resilient component 134 is forced by the first transmission component 131 to resiliently deform to push the second transmission component 133 to rotate along the first rotating direction R1. The stopping resilient component 135 is further configured to engage with the second transmission component 133 for stopping the second transmission component 133 from rotating along a second rotating direction R2 opposite to the first rotating direction R1 when the driving resilient component 134 is released to resiliently recover.

The third transmission component 136 is fixedly connected to the second transmission component 133, e.g., by a tightly fitting manner or an integrally forming manner. The third transmission component 136 is configured to be driven by the second transmission component 133 to rotate together with the second transmission component 133 when the second transmission component 133 rotates. The sliding component 137 is at least partially slidably disposed inside the third transmission component 136 and movably engaged with the third transmission component 136. The sliding component 137 is configured to be driven by the third transmission component 136 to slide relative to the second transmission component 133 along a first sliding direction S1 when the third transmission component 136 is driven by the second transmission component 133 to rotate along the first sliding direction S1 together with the second transmission component 133. The supporting component 138 includes a guiding portion 1381. The sliding component 137 passes through the guiding portion 1381, and the guiding portion 1381 is configured to guide the sliding component 137 to slide along the first sliding direction S1 without any rotation.

As shown in FIG. 2, the sliding component 137 is connected to the plunger 12. When the sliding component 137 is driven by the third transmission component 136 to slide along the first sliding direction S1, the plunger 12 is driven to slide relative to the reservoir 11 along the first sliding direction S1, so as to pump the drug out of the reservoir 11 to complete the drug injection.

Specifically, the first transmission component 131 can be an eccentric cam component. The second transmission component 133 can be a ratchet component. The third transmission component 136 can be a screw sleeve. The sliding component 137 can be a screw rod, and the driving component 132 can be an electric motor. However, the present invention is not limited to this embodiment. It depends on practical demands. For example, in another embodiment, the driving component can be a pneumatic motor.

Besides, in this embodiment, as shown in FIG. 3 to FIG. 9, the driving mechanism 13 further includes a reducer 139 coupled between the driving component 132 and the first transmission component 131. An input shaft and an output shaft of the reducer 139 can be respectively connected to the driving component 132 and the first transmission component 131. The reducer 139 can have various reduction ratios to control a rotating speed of the first transmission component 131, so as to control a sliding speed of the sliding component 137.

Specifically, the reducer 139 can be a gearbox. However, the present invention is not limited to this embodiment. It depends on practical demands. For example, in another embodiment, the reducer 139 can be a pulley and belt system. Alternatively, in another embodiment, the reducer can be omitted.

It should be noted that the aforementioned configuration can not only have small occupied space and achieve high power and high efficiency transmission but also achieve adjustment of a rotating speed of the second transmission component 133 by adjusting a reduction ratio between the driving component 132 and the first transmission component 131, e.g., a gear ratio of the reducer 139.

In order to make structure of the driving mechanism reasonably compact, as shown in FIG. 3 to FIG. 9, a first rotating axis A1 of the first transmission component 131 is parallel to and offset from a second rotating axis A2 of the second transmission component 133, and the first sliding direction S1 is parallel to an extending direction of the second rotating axis A2 of the second transmission component 133. The supporting component 138 includes a first supporting part 1382 and a second supporting part 1383 detachably assembled with the first supporting part 1382. The first transmission component 131, the second transmission component 133 and the third transmission component 136 are accommodated inside the supporting component 138 and located between the first supporting part 1382 and the second supporting part 1383. The driving resilient component 134 and the stopping resilient component 135 are integrally connected to each other to form an integral resilient structure. As shown in FIG. 7 and FIG. 9, the supporting component 138 further includes a mounting portion 1384 formed on the first supporting part 1382 and for mounting the integral resilient structure. In this embodiment, a channel 1385 is formed on the mounting portion 1384. The integral resilient structure passes through the channel 1385, and the driving resilient component 134 and the stopping resilient component 135 are partially exposed out of the mounting portion 1384. As shown in FIG. 7, the driving resilient component 134 is configured to be resiliently deformed by the first transmission component 131 along a first deforming direction D1 identical to the first rotating direction R1, and the stopping resilient component 135 is configured to be resiliently deformed by the second transmission component 133 along a second deforming direction D2 opposite to the first deforming direction D1. However, the present invention is not limited to this embodiment. For example, in another embodiment, the driving resilient component and the stopping resilient component can be separated from each other and located opposite to each other relative to the second transmission component.

Furthermore, in this embodiment, a rotating direction of the first transmission component 131 is identical to a rotating direction of the second transmission component 133. However, the present invention is not limited to this embodiment. For example, in another embodiment, the rotating direction of the first transmission component can be opposite to the rotating direction of the second transmission component, i.e., the first transmission component still can resiliently deform the driving resilient component along the first deforming direction for pushing the second transmission component to rotate along the first rotating direction even when the first transmission component rotates along the second rotating direction. In other words, the present invention can always ensure the drug injection no matter whether the rotating direction of the first transmission component is identical to the rotating direction of the second transmission component or not.

Besides, in order to achieve configuration of the guiding portion 1381 to guide the sliding component 137 to slide without any rotation when the third transmission component 136 rotatably drives the sliding component 137 to slide, as shown in FIG. 8 to FIG. 9, the guiding portion 1381 is formed on the first supporting part 1382 and includes a sliding through hole structure 13811. The sliding component 137 slidably passes through the sliding through hole structure 13811. A cross section of the sliding component 137 matches with a cross section of the sliding through hole structure 13811. The sliding component 137 includes two first arc parts 1371 opposite to each other, and two first flat parts 1372 opposite to each other and connected to the two first arc parts 1371. The sliding through hole structure 13811 includes two second arc parts 138111 opposite to each other, and two second flat parts 138112 opposite to each other and connected to the two second arc parts 138111. The two second arc parts 138111 and the two second flat parts 138112 are respectively corresponding to the two first arc parts 1371 and the two first flat parts 1372. An internal thread structure 1361 is formed on an inner periphery of the third transmission component 136, e.g., by plastic injection molding or insert molding, and an outer thread structure 13711 is formed on each of the first arc parts 1371 of the sliding component 137. The aforementioned configuration can ensure no rotation of the sliding component 137 during a sliding movement of the sliding component 137 by a cooperation of the sliding component 137 and the sliding through hole structure 13811, so as to meet a requirement of accurate drug dose delivery rate.

However, the structures of the sliding component and the guiding portion are not limited to this embodiment. For example, in another embodiment, the sliding component can include only one first arc part and one first flat part connected to the first arc part, and the sliding through hole structure can include only one second arc parts and one second flat part connected to the second arc part.

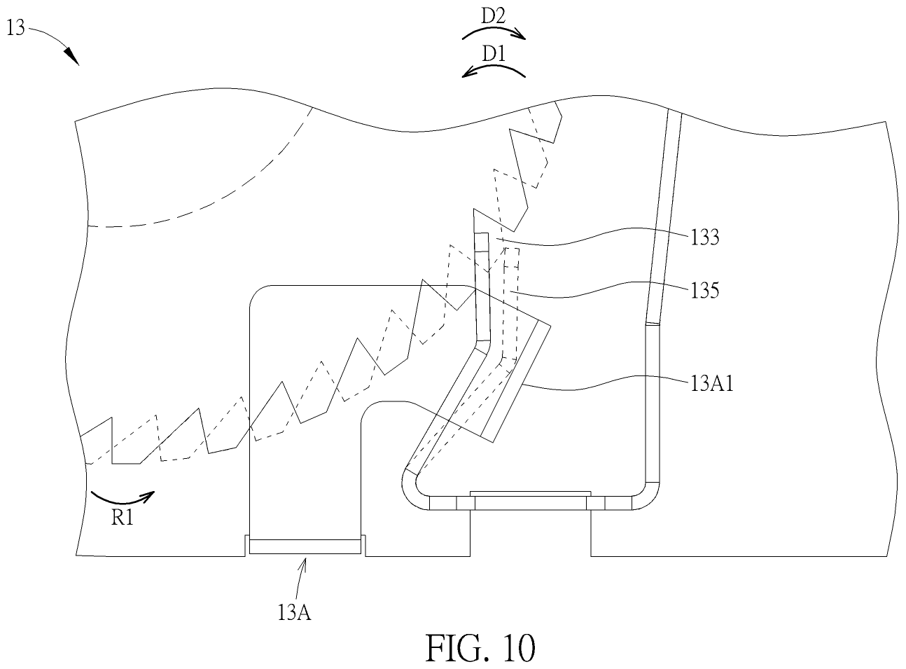

Please refer to FIG. 7 to FIG. 10. FIG. 10 is a partial enlarged diagram of the driving mechanism 13 according to the first embodiment of the present invention. As shown in FIG. 7 to FIG. 10, the driving mechanism 13 further includes a sensor 13A configured to sense a rotating movement of the second transmission component 133. In this embodiment, the sensor 13A includes an abutting component 13A1 disposed on the first supporting part 1382. The abutting component 13A1 is abutted by the stopping resilient component 135 when the stopping resilient component 135 is resiliently deformed by the second transmission component 133. Specifically, the abutting component 13A1 and the stopping resilient component 135 can respectively be two electrically conductive components electrically connected to a positive terminal and a negative terminal of an electric circuit. The electrical circuit can be closed for generating a first signal when the stopping resilient component 135 is resiliently deformed by the second transmission component 133 and abuts against the abutting component. The electrical circuit can be open for generating a second signal when the stopping resilient component 135 is released, e.g., separated away from the second transmission component 133, to resiliently recover and does not abut against the abutting component 13A1. However, the present invention is not limited to this embodiment. For example, in another embodiment, the sensor can be a non-contact sensor, e.g., a light sensor having a light receiver and a light emitter, and can be configured to generate the first signal when the stopping resilient component is resiliently deformed by the second transmission component to block or reflect light emitted from the light emitter.

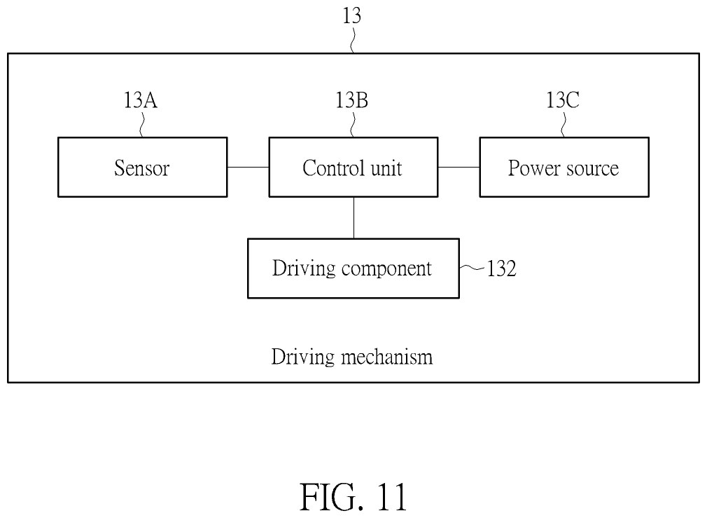

Please refer to FIG. 11. FIG. 11 is a functional block diagram of the driving mechanism 13 according to the first embodiment of the present invention. As shown in FIG. 11, the driving mechanism 13 further includes a control unit 13B and a power source 13C. The control unit 13B can be a circuit board electrically connected to the sensor 13A and the driving component 132 and configured to not only calculate a rotating speed, a number of rotation of the second transmission component 133 and/or a travel distance of the sliding component 137 according to the signals generated by the sensor 13A but also control the driving component 132 to actuate or stop a rotating movement of the first transmission component 131 and/or to control the rotating speed or a rotating direction of the first transmission component 131. The power source 13C can be a battery electrically connected to the control unit 13B and configured to provide electricity to the control unit 13B.

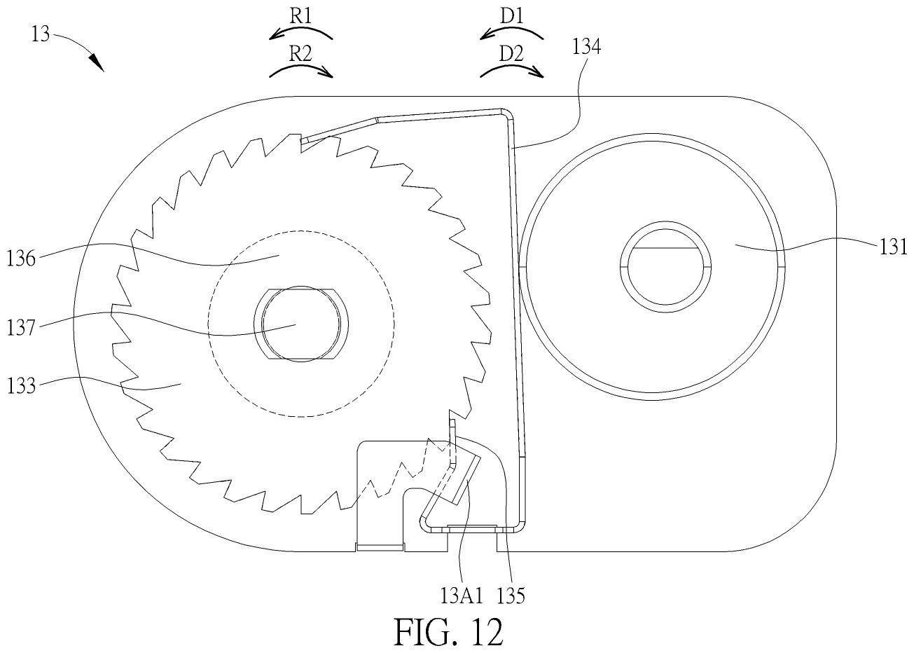

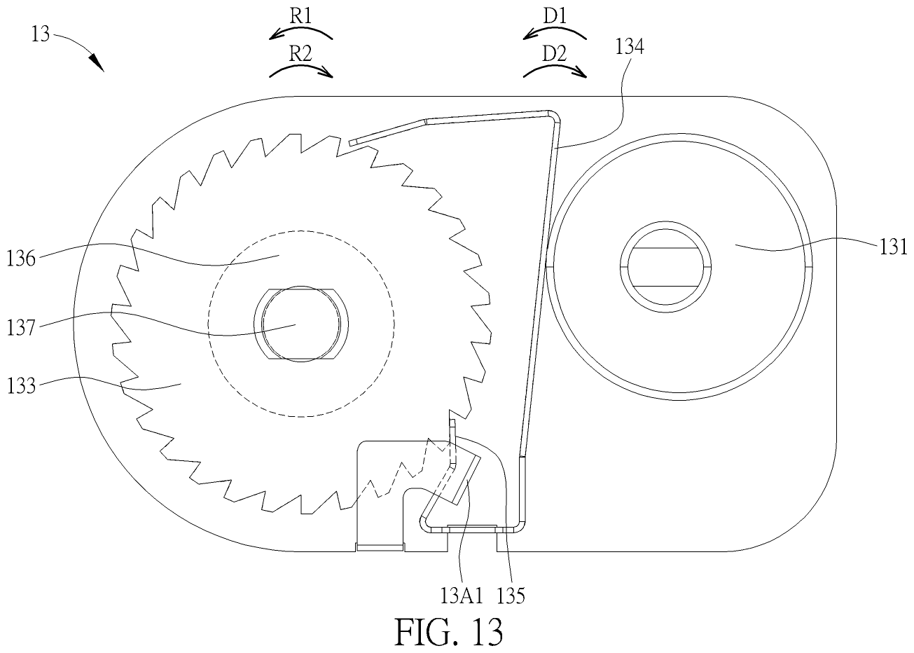

Detailed description for operational principle of the auto-injector 1 is provided as follows. Please further refer to FIG. 2, FIG. 5, FIG. 7, FIG. 10, FIG. 12 and FIG. 13. FIG. 12 and FIG. 13 are diagrams of the driving mechanism 13 in different states according to the first embodiment of the present invention. As shown in FIG. 2, FIG. 5, FIG. 7, FIG. 10, FIG. 12 and FIG. 13, after the auto-injector 1 is attached on a patient's body, the driving component 132 can be controlled by the control unit 13B to drive the first transmission component 131 to rotate around the first rotating axis A1 along the first rotating direction R1 with the reducer 139. When the first transmission component 131 is driven to rotate around the first rotating axis A1 along the first rotating direction R1, the driving resilient component 134 is forced by the first transmission component 131 to resiliently deform along the first deforming direction D1 to push the second transmission component 133 to rotate along the first rotating direction R1 and then released to resiliently cover along the second deforming direction D2 repeatedly. When the second transmission component 133 rotates around the second rotating axis A2 along the first rotating direction R1, the third transmission component 136 rotates around the second rotating axis A2 along the first rotating direction R1 together with the second transmission component 133, so that the sliding component 137 is driven to slide relative to the third transmission component 136 along the first sliding direction S1 without any rotation, so as to drive the plunger 12 to slide relative to the reservoir 11 along the first sliding direction S1 for pumping drug out of the reservoir 11.

Specifically, in each rotation cycle of the first transmission component 131, the driving resilient component 134 is moved from a position as shown in FIG. 13 to a position as shown in FIG. 12 and then back to the position as shown in FIG. 13. When the driving resilient component 134 is moved from the position as shown in FIG. 13 to the position as shown in FIG. 12, the second transmission component 133 is driven by the driving resilient component 134 to rotate along the first rotating direction R1 at a predetermined rotation angle. During the aforementioned process, as shown in FIG. 10, the stopping resilient component 135 is resiliently deformed along the second deforming direction D2 by the second transmission component 133 and then released to recover along first deforming direction D1. Afterwards, when the driving resilient component 134 is recovered from the position as shown in FIG. 12 back to the position as shown in FIG. 13, the recovered stopping resilient component 135 engages with the second transmission component 133 for preventing the recovered driving resilient component 134 from driving the second transmission component 133 to rotate along the second rotating direction R2, so that the second transmission component 133 is stopped.

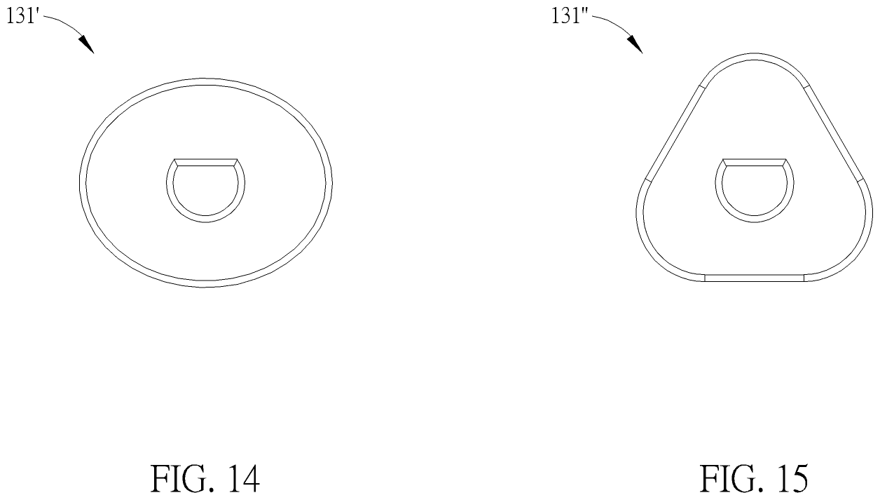

Understandably, a shape of the first transmission component 131 can be determined according to practical demands, and it decides a driving frequency of the driving resilient component 134 for driving the second transmission component 133 to rotate along the first rotating direction R1 in each rotation cycle of the first transmission component 131. For example, please refer to FIG. 14 and FIG. 15. FIG. 14 is a diagram of a first transmission component 131′ according to a second embodiment of the present invention. FIG. 15 is a diagram of a first transmission component 131″ according to a third embodiment of the present invention. As shown in FIG. 14, in the second embodiment, the first transmission component 131′ can be an ellipse cam component, which can push the driving resilient component twice in each rotation cycle of the first transmission component 131′. As shown in FIG. 15, in the third embodiment, the first transmission component 131″ can be a triangular cam component, which can push the driving resilient component three times in each rotation cycle of the first transmission component 131″.

Besides, understandably, a circular pitch of the second transmission component 133 also can be determined according to practical demands, which decides the predetermined rotation angle of the second transmission component 133 in each rotation cycle of the first transmission component 131.

In contrast to the prior art, in the present invention, the driving mechanism not only has compact structure and high power and high efficiency transmission but also achieves a long sliding distance and a slow sliding speed of the sliding component and prevents any rotation of the sliding component during a sliding movement of the sliding component. Therefore, the present invention can meet requirements of small volume, high driving power, long driving distance, long injecting period and accurate drug dose delivery rate.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A driving mechanism for driving a plunger of an auto-injector to slide relative to a reservoir of the auto-injector, the driving mechanism comprising:
a first transmission component;
a driving component coupled to the first transmission component and for driving the first transmission component to rotate;
a second transmission component rotatably disposed apart from the first transmission component;
a driving resilient component arranged between the first transmission component and the second transmission component, the driving resilient component being forced by the first transmission component to resiliently deform to push the second transmission component to rotate along a first rotating direction or released to resiliently cover;
a stopping resilient component disposed adjacent to an outer periphery of the second transmission component, the stopping resilient component being forced by the second transmission component to resiliently deform when the driving resilient component is forced by the first transmission component to resiliently deform to push the second transmission component to rotate along the first rotating direction, the stopping resilient component engaging with the second transmission component for stopping the second transmission component from rotating along a second rotating direction opposite to the first rotating direction when the driving resilient component is released to resiliently recover;
a third transmission component fixedly connected to the second transmission component, the third transmission component being driven by the second transmission component to rotate together with the second transmission component when the second transmission component rotates;
a sliding component at least partially slidably disposed inside the third transmission component and movably engaged with the third transmission component, the sliding component being connected to the plunger, the sliding component being driven by the third transmission component to slide relative to the second transmission component along a first sliding direction when the third transmission component is driven by the second transmission component to rotate along the first sliding direction together with the second transmission component; and
a supporting component comprising a guiding portion, the sliding component passing through the guiding portion, and the guiding portion being configured to guide the sliding component to slide along the first sliding direction without rotation.

2. The driving mechanism of claim 1, further comprising a sensor configured to sense a rotating movement of the second transmission component.

3. The driving mechanism of claim 2, wherein the sensor comprises an abutting component, the abutting component is abutted by the stopping resilient component when the stopping resilient component is resiliently deformed by the second transmission component.

4. The driving mechanism of claim 1, wherein the driving resilient component and the stopping resilient component are integrally connected to each other to form an integral resilient structure.

5. The driving mechanism of claim 4, wherein the supporting component further comprises a mounting portion for mounting the integral resilient structure.

6. The driving mechanism of claim 5, wherein a channel is formed on the mounting portion, the integral resilient structure passes through the channel, and the driving resilient component and the stopping resilient component are partially exposed out of the mounting portion.

7. The driving mechanism of claim 1, wherein the driving resilient component is resiliently deformed by the first transmission component along a first deforming direction identical to the first rotating direction, and the stopping resilient component is resiliently deformed by the second transmission component along a second deforming direction opposite to the first deforming direction.

8. The driving mechanism of claim 1, wherein the first transmission component, the second transmission component and the third transmission component are accommodated inside the supporting component.

9. The driving mechanism of claim 1, wherein a first rotating axis of the first transmission component is parallel to a second rotating axis of the second transmission component, and the first sliding direction is parallel to an extending direction of the second rotating axis of the second transmission component.

10. The driving mechanism of claim 1, wherein the first transmission component is a cam component, the second transmission component is a ratchet component, the third transmission component is a screw sleeve, the sliding component is a screw rod, and the driving component is an electric motor.

11. The driving mechanism of claim 1, further comprising a reducer coupled between the driving component and the first transmission component.

12. The driving mechanism of claim 11, wherein the reducer is a gearbox.

13. The driving mechanism of claim 1, wherein the guiding portion comprises a sliding through hole structure, the sliding component slidably passes through the sliding through hole structure, a cross section of the sliding component matches with a cross section of the sliding through hole structure, the sliding component comprises at least one first arc part and at least one first flat part connected to the at least one first arc part, the sliding through hole structure comprises at least one second arc part and at least one second flat part connected to the at least one second arc part, and the at least one second arc part and the at least one second flat part are arranged respectively corresponding to the at least one first arc part and the at least one first flat part.

14. The driving mechanism of claim 13, wherein an internal thread structure is formed on an inner periphery of the third transmission component, and an external thread structure is formed on the at least one first arc part of the sliding component.

15. An auto-injector comprising:
a reservoir;
a plunger slidably disposed inside the reservoir; and
a driving mechanism for driving the plunger to slide relative to the reservoir, the driving mechanism comprising:
a first transmission component;
a driving component coupled to the first transmission component and for driving the first transmission component to rotate;
a second transmission component rotatably disposed apart from the first transmission component;
a driving resilient component arranged between the first transmission component and the second transmission component, the driving resilient component being forced by the first transmission component to resiliently deform to push the second transmission component to rotate along a first rotating direction or released to resiliently cover;
a stopping resilient component disposed adjacent to an outer periphery of the second transmission component, the stopping resilient component being forced by the second transmission component to resiliently deform when the driving resilient component is forced by the first transmission component to resiliently deform to push the second transmission component to rotate along the first rotating direction, the stopping resilient component engaging with the second transmission component for stopping the second transmission component from rotating along a second rotating direction opposite to the first rotating direction when the driving resilient component is released to resiliently recover;
a third transmission component fixedly connected to the second transmission component, the third transmission component being driven by the second transmission component to rotate together with the second transmission component when the second transmission component rotates;
a sliding component at least partially slidably disposed inside the third transmission component and movably engaged with the third transmission component, the sliding component being connected to the plunger, the sliding component being driven by the third transmission component to slide relative to the second transmission component along a first sliding direction when the third transmission component is driven by the second transmission component to rotate along the first sliding direction together with the second transmission component; and
a supporting component comprising a guiding portion, the sliding component passing through the guiding portion, and the guiding portion being configured to guide the sliding component to slide along the first sliding direction without rotation.

16. The auto-injector of claim 15, wherein the driving mechanism further comprising a sensor configured to sense a rotating movement of the second transmission component.

17. The auto-injector of claim 16, wherein the sensor comprises an abutting component, the abutting component is abutted by the stopping resilient component when the stopping resilient component is resiliently deformed by the second transmission component.

18. The auto-injector of claim 15, wherein the driving resilient component and the stopping resilient component are integrally connected to each other to form an integral resilient structure, and the supporting component further comprises a mounting portion for mounting the integral resilient structure.

19. The auto-injector of claim 18, wherein a channel is formed on the mounting portion, the integral resilient structure passes through the channel, and the driving resilient component and the stopping resilient component are partially exposed out of the mounting portion.

20. The auto-injector of claim 15, wherein the driving resilient component is resiliently deformed by the first transmission component along a first deforming direction identical to the first rotating direction, and the stopping resilient component is resiliently deformed by the second transmission component along a second deforming direction opposite to the first deforming direction.
